(19) United States
(12) Patent Application Publication
(10) Pub. No.: US 2002/0064538 A1
(43) Pub. Date: May 30, 2002

(54) Method of controlling elasticity of skin and composition for the same

(76) Inventors: Jin-Ho Chung, Seoul (KR); Jin-Young Seo, Seoul (KR); Hye-Ryung Choi, Seoul (KR)

Correspondence Address: Douglas W. Sprinkle, Gifford, Krass, Groh, Sprinkle,, Anderson & Citkowski, P.C., 280 N. Old Woodward Avenue, Suite 400, Birmingham, MI, 48009, US

(21) Appl. No.: 09/780,228

(22) Filed: Feb. 9, 2001

(30) Foreign Application Priority Data
Nov. 30, 2000 (KR) 2000-71823

(51) Int. Cl. A61K 35/78; A61K 38/06; A61K 31/198; A61K 31/375

(52) U.S. Cl. 424/401; 424/729; 424/757; 514/18; 514/474; 514/562

(57) ABSTRACT

The present invention relates to a method for controlling elasticity of skin and compositions for the same, and more particularly, to a method for controlling elasticity of skin comprising the step of using a compound that inhibits or induces expression of tropoelastin mRNA or protein in keratinocytes. The present invention provides compositions for maintaining elasticity of skin by controlling tropoelastin mRNA or protein within human skin. The method for controlling elasticity of skin and compositions thereof regulates tropoelastin expression in keratinocytes, maintaining elasticity of skin and inhibiting production of elastotic material.

### BACKGROUND OF THE INVENTION

(a) Field of the Invention

The present invention relates to a method for controlling elastin expression of skin and compositions for the same, and more particularly, to a method for controlling elasticity of skin comprising the step of using a compound that inhibits or induces expression of tropoelastin mRNA or protein in keratinocytes.

(b) Description of the Related Art

Elastin fibers are structural elements of connective tissues and contribute to the elasticity of the skin in the extracellular space of connective tissue. Elastin fibers are synthesized as the polypeptide chain tropoelastin, and are cross-linked between tropoelastins in the extracellular space. Thus, elastin consists of tropoelastin molecules cross-linked to give two-dimensional or three-dimensional elasticity.

Several cell types have been shown to produce tropoelastin: fibroblasts (Sephel G C, Davidson J M. J Invest Dermatol 86:279-285, 1986), smooth muscle cells (Hinek A, Thyberg J. J Ultrastruct Res 60: 12-20 1977: Hayashi A, Wachi H. Tajima S: Biochem Biophys Acta 1244:325-330, 1995), endothelial cells (Mecham R P, Madaras J, McDonald J A, Ryan U J Cell Physiol 116:282-288, 1983), keratinocytes (Kajiya H, Tanaka N, Inazumi T, Seyama Y, Tajima S, Ishibashi A. J Invest Dermatol 109:641-644, 1997), and modified or malignant epithelial cells (Krishnan R, Cleary E G. Cancer Res 50:2164-2171, 1990; Starcher B, Pierce R, Hinek A. J Invest Dermatol 112:450-455, 1999). In normal skin, it has been assumed that the cells responsible for elastin synthesis are mainly fibroblasts in the dermis, and under normal circumstances keratinocytes are not considered as elastin-producing cells.

Skin aging can be divided into two areas, intrinsic (chronological) aging and photoaging (Gilchrest B A: Skin aging and photoaging: an overview. J Am Acad Dermatol 21:610-613. 1989). The histologic findings from intrinsic aging show a general decrease in the extracellular matrix with reduced elastin and a disintegration of elastic fibers (Braverman I M, Fonferko E: Studies in cutaneous aging. I. J Invest Dermatol 78: 434-443, 1982). Photoaging means that skin changes for the worse in appearance and function, and especially it becomes wrinkled. Also, the histologic findings of photoaged skin show the most prominent feature, referred to as solar elastosis, which is characterized by the accumulation of dystrophic elastotic material in the reticular dermis (Montagna W, Kirchner S, Carlisle K: Histology of sun-damaged shin. J Am Acad Dermatol 21:907-918,1989; Warren R, Gartsetin V, Kligman A M, Montagna W, Allendorf R A, Ridder G M: Age, sunlight, and facial skin: a histologic and quantitative study. J Am Acad Dermatol 25:751-760,1991; Taylor C R, Stern R S, Leyden J J, Gilchrest B A: Photoaging/photodamage and photoprotection. J Am Acad Dermatol 22:1-15, 1990; Mera S L, Lovell C R, Jones R R, Davies J D: Elastic fibres in normal and sun-damaged skin: an immunohistochemical study. Br J Dermatol 117:21-27,1987). Little is known about the mechanisms leading to the accumulation of elastotic material in photoaged skin, although this material stains strongly with elastic tissue stains (Chen V L, Fleischmajer R, Schwartz E, Palaia M, Timpl R: Immunochemistry of elastotic material in sun-damaged skin. J Invest Dermatol 87:334-337,1986; Werth V P, Kalathil E, Jaworsky C: The distribution of microfibrillar and elastic proteins on dermal elastic fiber in development and photoaging. Photochem Photobiol 63:308-313, 1996).

Ultraviolet irradiation has been directly shown to upregulate tropoelastin gene expression both in vivo and in vitro (Uitto J, Brown D B, Gasparro F P, Bernstein E F: Molecular aspects of photoaging. Eur J Dermatol 7:210-214, 1997). This accumulation of elastotic material may be associated with increased elastin production by ultraviolet irradiation in photoaged skin.

Solar radiation reaching the earth's surface that effects and enables various animals, including humans, comprises ultraviolet (UV), visible and infrared (IR). UV radiation is generally divided into UVA (320-400 nm), UVB (290-320 nm), and UVC (<290 nm). UVA radiation generates direct sunburn, UVB radiation generates indirect sunburn, cutaneous cancer and erythema, and UVC radiation is blocked from reaching the earth's surface by stratospheric ozone.

Elastin has been used as a protection drug to improve the appearance of skin that expresses little elastin or has accumulated elastotic materials (U.S. Pat. No. 5,575,994). But an essential method of protecting skin may be achieved by controlling the expression of elastin and the accumulation of elastotic materials.

### SUMMARY OF THE INVENTION

It is an object of the present invention to provide a method for controlling elastin expression of skin.

It is an object to provide a composition for controlling elastin expression of skin.

It is an object to provide a method for preventing photoaging of skin.

These and other objects may be achieved by a method for controlling elasticity of skin comprising the step of using a composition that inhibits or induces expression of tropoelastin mRNA or protein in keratinocytes.

The present invention provides a composition for controlling elasticity of skin involved in inhibition or induction of tropoelastin mRNA or protein expression in human epithelial cells, keratinocytes.

### BRIEF DESCRIPTION OF THE DRAWINGS

FIG. 1 is an in situ hybridization slide showing the time dependent changes of tropoelastin mRNA expression after UVB irradiation.

FIG. 2 is a picture showing the time dependent changes of tropoelastin mRNA expression determined by RT-PCR after UVB irradiation.

FIG. 3 shows a method of Laser assisted microdissection and expression of tropoelastin mRNA determined by RT-PCR in keratinocytes.

FIG. 4 is a picture showing expression level of tropoelastin mRNA after UVB irradiation investigated by the RT-PCR method in cultured keratinocytes.

FIG. 5 is a picture showing expression of tropoelastin mRNA and protein in keratinocyte and fibroblast according to aging.

FIG. 6 is a picture showing expression of tropoelastin mRNA and protein in sun-protected skin from the upper-inner arm, and sun-exposed skin from the forearm.

FIG. 7 is a picture showing expression of tropoelastin in retinoic acid treated aged skin.

### DETAILED DESCRIPTION OF THE PREFERRED EMBODIMENTS

The present invention will now be explained in more detail.

Photoaging is characterized by the accumulation of elastotic material in the reticular dermis.

Elastin is a structural element that is synthesized in skin tissues. But elastin is transformed to dystrophic elastotic material by UV irradiation, and elastotic material is accumulated at the dermis. Thus the hard elastotic material causes aging conditions such as skin-drooping. Although little is known about the mechanism leading to the accumulation of elastotic material in photoaged skin, various proteinase produced from inflammatory cells and other solar dependent specific mechanisms may contribute to elastosis.

In the present invention, it is demonstrated that keratinocytes express tropoelastin mRNA and protein after UV irradiation although they do not express in normal conditions and photoaging can be prevented by regulation of tropoelastin overexpression in keratinocytes. Also, elasticity of skin can be maintained by controlling tropoelastin expression in keratinocytes of aged skin.

In the present invention, it was shown that UVB irradiation induced tropoelastin mRNA expression in human epithelial cells such as keratinocytes. Tropoelastin expression in keratinocytes induces elastin production of photoaged skin and causes accumulation of elastotic material.

Also, the expression level of tropoelastin mRNA according to aging was determined. In young skin, tropoelastin mRNA was strongly expressed in the dermal fibroblasts but not in the keratinocytes. However, in aged sun-protected skin, there was no expression in the keratinocytes and there was a dramatic decrease in tropoelastin mRNA expression in the fibroblasts, by an average of 40%, when compared with young skin. Therefore, in intrinsic aging, tropoelastin mRNA was expressed little in keratinocytes regardless of aging, and the young skin actively expressed tropoelastin mRNA in fibroblast, when compared with aging skin.

Also, it was further confirmed that UV irradiation induced tropoelastin mRNA and protein in keratinocytes. Tropoelastin mRNA expression in keratinocytes and fibroblasts was observed with sun-exposed skin and sun-protected-skin. As a result, minimal amounts of tropoelastin mRNA in the fibroblasts were detected in the sun-protected skin and there was no tropoelastin mRNA expression in the keratinocytes. While in sun-exposed skin, tropoelastin mRNA expression increased significantly not only in the fibroblasts but also in the keratinocytes, when compared with those of sun-protected skin. Therefore, UV irradiation induces expression of tropoelastin mRNA, especially in keratinocytes.

Also, the present invention provides a method for controlling elasticity of skin including inhibiting or inducing tropoelastin mRNA expression in keratinocytes, i.e. with controlling tropoelastin mRNA expression in keratinocytes, it can maintain elasticity of skin. The method for keeping elasticity of skin divides to an inhibitory method of elastin production by using a composition of suppressing tropoelastin mRNA or protein expression at keratinocytes, and a facilitating method of elastin production by using a composition for inducing tropoelastin mRNA or protein expression at keratinocytes. Also, the inhibitory method of tropoelastin mRNA or protein expression includes preventing photoaging while inhibiting accumulation of elastotic material by UV radiation.

Moreover, the present invention provides a composition for controlling tropoelastin mRNA and protein expression. It is preferred that a composition for inhibiting/inducing tropoelastin mRNA is selected from the group consisting of natural extracts, antioxidants, hormones, retinoid, vitamins and their mixture. The natural extracts are preferably green tea extract and soybean extract, the antioxidants are preferably vitamin C, N-acetylcystein (NAC) and glutathione, the hormones are preferably estrogen, DHEA (dehydroepiandrosterone) and testosterone. Also, the vitamins are preferably vitamin C, tocopherol, and vitamin D. It is preferred that the retinoid is selected from the group consisting of isomers of retinoic acid, tretinoin, retinol, retinaldehydes, their salt compounds and their ester compounds. It is preferably that the composition of the present invention further includes 0.001 wt % to 50 wt % of the composition for controlling elasticity of skin and 50 wt % to 99.99 wt % of pharmaceutically acceptable material.

The composition for controlling elasticity of skin can be used for medical supplies or cosmetics, and formulation of a composition of the present invention is preferably adjusted for use. In the case of applying it to medical supplies, it is preferable to formulate as a solution, gel, and cream. In the case of applying it to cosmetics, it is preferable to use it with a cleanser, lotion and pack, and formulate it as a solid, solution, cream, gel, milky liquid and so on.

The present invention will be explained in more detail with reference to the following Examples. However, the following Examples are to illustrate the present invention and the present invention is not limited to them.

### EXAMPLE 1

Tropoelastin mRNA Expression in Keratinocytes with UV

UVB Irradiation at Skin

Korean adults, volunteers without current or prior skin disease, were studied in vivo. A Waldmann UV-800 (Waldmann Co., Villingen-Schwenningen, Germany) phototherapy device, including F75/85W/UV21 fluorescent sunlamps, served as the UVB source, having an emission spectrum of between 285-350 nm (peak at 310-315 nm). Irradiation at the skin surface was measured with a Waldmann UV meter (Model No. 585100; Waldmann Co., Villingen-Schwenningen, Germany). The skin of the buttocks was irradiated with UVB and the dose that caused minimal erythema was determined 24 hours after irradiation. The buttock skin was irradiated with 2 MED of UVB and specimens of both irradiated and non-irradiated skin were obtained from each subject 24, 48, and 72 hours after irradiation (n=5).

In situ Hybridization

Digoxigenin-containing sense and antisense riboprobes to detect human tropoelastin mRNA were synthesized using T3 and T7 RNA polymerases (Fisher G J, Wang Z, Datta S C, Varani J, Kang S, Voorhees J J: Pathophysiology of premature skin aging induced by ultraviolet light. New Engl J Med 337:1419-1428, 1997). The 0.8 kb digoxigenin-labeled RNA probe was hydrolyzed in a solution of 30 mM sodium carbonate and 20 mM sodium bicarbonate at 60° C. In situ hybridization was performed on 8 μm sections as described in detail elsewhere (Fisher G J, Wang Z, Datta S C, Varani J, Kang S, Voorhees J J: Pathophysiology of premature skin aging induced by ultraviolet light. New Engl J Med 337:1419-1428, 1997). All samples were treated with proteinase K and were washed in a 0.1 M triethanolamine buffer containing 0.25% acetic anhydride. After hybridization at 52° C., the slides were washed under stringent conditions, including treatment with RNase A to remove the unhybridized probe. Hybridization signals were detected immunohistochemically with the use of an alkaline phosphatase-conjugated antidigoxigenin antibody.

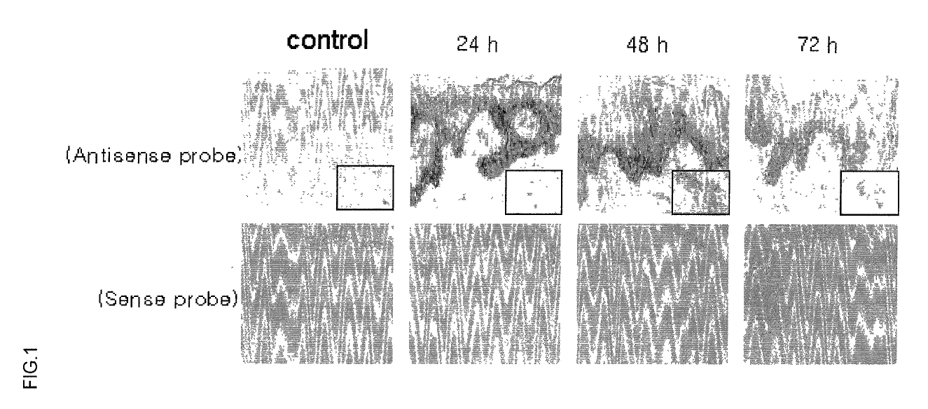

FIG. 1 is an in situ hybridization slide showing the time-dependent changes of expression level of tropoelastin mRNA according to hours after UVB irradiation. The upper part of each picture shows epidermal tissue such as keratinocytes and the inserted box located at the right-lower end of each picture shows fibroblast of identical epidermal tissue. In the control group, tropoelastin mRNA was not detectable in the keratinocytes of non-irradiated skin. Twenty-four hours after exposure to ultraviolet irradiation the expression of tropoelastin mRNA was induced, and after 48 hours it reached a maximum in all keratinocytes throughout the epidermis. At longer times (72 hours post-UVB), the expression of tropoelastin mRNA decreased gradually. However, ultraviolet irradiation tended to decrease tropoelastin mRNA expression in the fibroblasts 24 hours post-UVB (FIG. 1a insert), after which there was a recovery of tropoelastin mRNA to normal levels or higher than normal levels at 48 and 72 hours post-UVB.

RT-PCR

The time dependent changes of tropoelastin mRNA expression after UVB irradiation were determined by RT-PCR. The epidermis was completely separated from the dermis in the 20 mM ribonucleoside vanadyl complex at 65° C. for 1.5 minutes.

The total RNA was isolated from the epidermis using a Trizol reagent. 3 ug of the total RNA extracted from the epidermis was reverse-transcribed using a 1st strand cDNA synthesis kit for RT-PCR (Roche Diagnostics GmbH, Germany). The resulting specific cDNA fragments were amplified with 2.5 U of Taq polymerase (Roche Diagnostics GmbH, Germany) in the presence of 20 pmol downstream primer (5′-ACCTGGGACAACTGGAATCC-3′) and upstream primer (5′-AAAGCAGCAGCAAAGTTCGG-3′).

The primers specifically amplified a 276 base pair fragment corresponding to the region between base 780 and 1068 of human elastin mRNA (Indik Z, Yeh H, Ortenstein-Goldstein N, et al: Alternative splicing of human elastin mRNA indicated by sequence analysis of cloned genomic and complementary DNA, Proc Natl Acad Sci USA 84:5680-5684, 1987). Amplification was performed in a thermacycler for 34 cycles of 1 minute at 94° C. for denaturing, 1 minute at 60° C. for re-annealing and 3 minutes at 72° C. for extension. To evaluate the concentration of RNA in each sample, GAPDH mRNA was amplified in the presence of a 20 pmol sense primer (5′ATTGTTGCCATCAATGACCC-3′) and an antisense primer (5′AGTAGAGGCAGGGATGATGT-3′) by RT-PCR with an optimized number of 27 cycles.

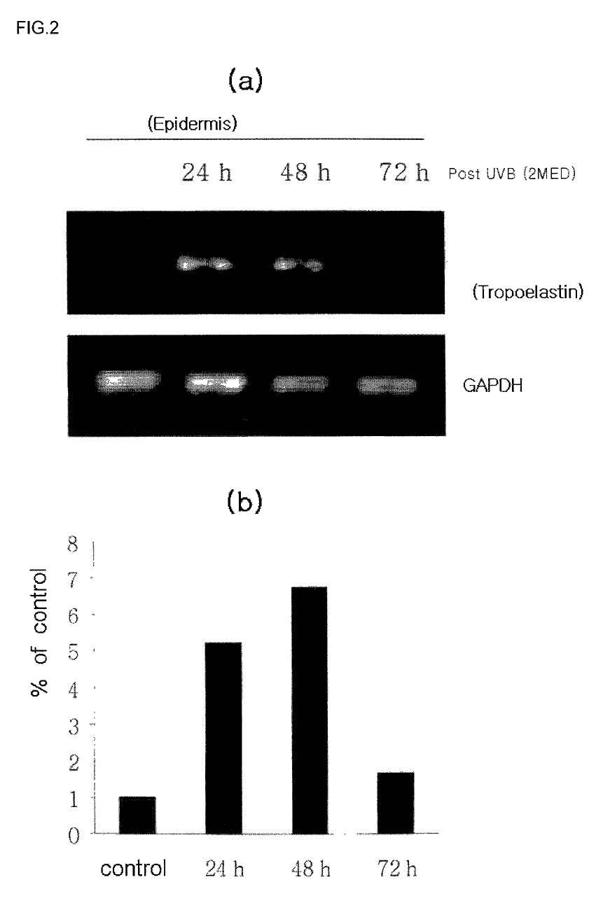

FIG. 2 shows the time-dependent changes of tropoelastin mRNA expression after UVB irradiation investigated by the RT-PCR method. There was very low tropoelastin mRNA expression in the control epidermis. However, the tropoelastin mRNA level in the epidermis was increased at 24 and 48 hours post-UV and the level then decreased to the near normal level at 72 hours post-UVB.

Also, the PCR product was isolated, sequenced, and found to be identical to the tropoelastin cDNA fragment.

In addition, to confirm expression of tropoelastin mRNA in keratinocytes, the epidermis was carefully microdissected to avoid contamination with dermal components using laser assisted microdissection. RT-PCR was then performed using the total RNA extracted from the captured epidermal tissues.

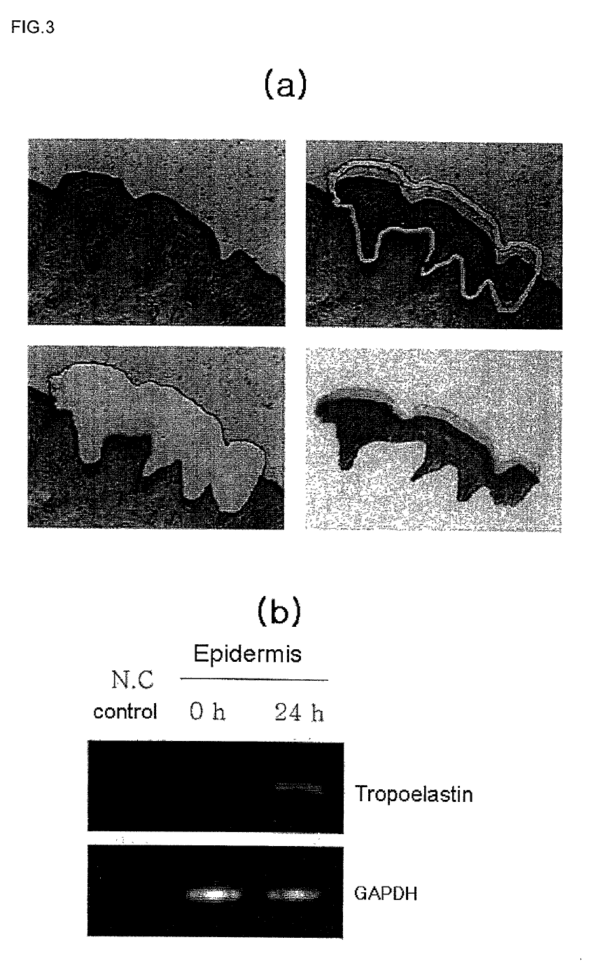

The method of Laser assisted microdissection is as follows, and is displayed in FIG. 3. Sections of skin (4 um) were mounted onto a 1-2 um thick supporting membrane (P.A.L.M. Co., Wolfratshausen, Germany), air dried, and fixed in 70% ethanol. After fixation, the slides were transferred to DEPC-water and then stained with Mayer's hematoxylin and eosin. The slides were washed with ethanol followed by xylene, then air dried before microdissection.

The UV-laser microbeam (P.A.L.M. Co., Wolfratshausen, Germany) used for microdissection consisted of a high-beam precision nitrogen laser (wavelength 337 nm), which was coupled to an inverted microscope (Axiovert 135; Zeiss, Jena, Germany) via the epifluorescence illumination path. The microscope stage and micromanipulator were digitally controlled and moved by a computer mouse. The epidermis was microdissected from the dermis precisely following its irregular shape. The entire membrane-tissue stack was ejected with one single laser shot and captured in the collector. After microdissection of each specimen, the mineral oil-coated cap containing the captured epidermis was placed in a microtube. RNA was extracted using a Trizol reagent (Gibco BRL, Gaithersburg, Md.) according to manufacturer's recommendations. Genomic DNA was digested to completion with DNase I (RNase-free). The RNA was then purified by phenol/chloroform extraction and ethanol.

FIG. 3 shows a method for microdissecting epidermis from dermis and the expression level of tropoelastin mRNA investigated by the RT-PCR method. There was very low tropoelastin mRNA expression in the control epidermis, and epidermal tropoelastin mRNA expression was increased at 24 hours post-UV.

Expression of Tropoelastin mRNA In vitro

Human epidermal keratinocytes were obtained from the foreskins of healthy humans with an average age of between 20 and 29 years. The keratinocytes were grown in Keratinocyte growth media (Bio Whittaker Co., Walkersville, Md.) in a humidified atmosphere of 5% CO₂ at 37° C., were passaged after reaching 60% to 70% confluence, and were used at passage 3.

The keratinocytes cultured in monolayer were irradiated with UVB. After 24 hours post UV irradiation, total RNA was isolated both from keratinocytes treated with UVB irradiation and those not treated, and was reverse-transcribed. To investigate whether the culture methodology had an effect on tropoelastin mRNA expression, keratinocytes were cultured three-dimensionally on a collagen matrix without fibroblasts.

The collagen matrix was prepared by rapidly mixing eight volumes of collagen type 1 solution (Nitta Gelatin, Tokyo, Japan) with one volume of a tenfold concentration of DMEM and one volume of sodium bicarbonate (22 mg/ml). Aliquots (0.3 ml) of the collagen solution mixture were plated into a 12 mm polycarbonate filter chamber (3.0 um Millicell-pc; Millipore Co., Bedford, Mass.) in six well culture dishes. One hour later, skin keratinocytes were seeded at a density of 5×10⁵ cells/Millicell-pc on top of the collagen matrix, and cultured in a submerged state for 7 days, then in an air-liquid interface for 7 days. Cultures were maintained with E-medium consisting of a 3:1 mixture of DMEM and Ham's nutrient mixture F12 medium supplemented with 10% fetal bovine serum, 5 ug/ml insulin, 1×10₋₁₀ M cholera toxin, 0.4 ug/ml hydrocortisone, 5 ug/ml transferrin, and 1×10⁻¹¹ M triiodothyronine.

The keratinocytes cultured on collagen gel were irradiated with UVB. After 24 hours post UV irradiation, total RNA was isolated both from keratinocytes treated with UVB irradiation and those not treated, and was reverse-transcribed.

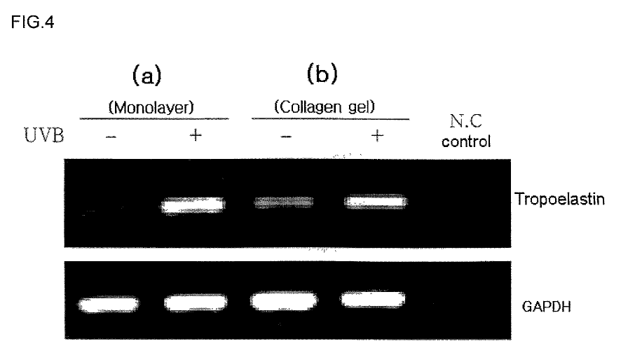

FIG. 4 shows the expression level of tropoelastin mRNA after UVB irradiation investigated by the RT-PCR method in cultured keratinocytes. The “a” shows the expression level of tropoelastin mRNA investigated by the RT-PCR method in the keratinocytes culture in monolayer and “b” shows the expression level of tropoelastin mRNA after UVB irradiation investigated by the RT-PCR method in keratinocytes cultured three-dimensionally on a collagen matrix. In “a”, tropoelastin mRNA expression could be detected in non-irradiated keratinocytes (a−), and was increased at 24 hours post-UVB (a+). Also, in “b”, keratinocytes cultured on the collagen gel showed low tropoelastin mRNA expression (b−) and showed increased tropoelastin mRNA expression after UVB irradiation (b+).

### EXAMPLE 2

Expression of Tropoelastin According to Aging

Specimens of both young (20-29 years) and aged (>70 years) buttock skin were obtained and both sets of skin were analyzed by in situ hybridization, northern blot and immunohistochemical stain.

In situ hybridization was generated as described in Example 1, and northern blot was performed to quantify tropoelastin mRNA. The total RNA was isolated and was electrophoresed through 1% formaldehyde gel and transferred onto a Hybond-N membrane (Amersham, Arlington Heights, Ill.) using a turboblotter (Schleicher and Schuell, Keene, N.H.) downward capillary transfer system. The CDNA probes were prepared by labeling the fragments of human tropoelastin (0.8 kb) and 36B4 (0.7 kb) with [α-32P]dCTP by using a Prime-It II kit (Stratagene, La Jolla, Calif.). The northern blot was analyzed by hybridization (Indik Z, Yeh H, Ortenstein-Goldstein N, et al: Alternative splicing of human elastin mRNA indicated by sequence analysis of cloned genomic and complementary DNA, Proc Natl Acad Sci USA 84:5680-5684, 1987).

The immunohistochemical staining method follows. 8 mm punch biopsy specimens from volunteers were placed immediately into a cryomatrix (Shandon, Pittsburgh, USA) and carried to the deep freezer (−70° C.). Serial sections of 8 mm thickness were mounted onto silane coated slides (Dako, Glostrup, Denmark). Acetone-fixed frozen sections were stained for polyclonal anti-human tropoelastin antibody (Elastin Products Company, Owensville, Mo.) analysis. Antibodies for tropoelastin, diluted 1:1600, were applied for 1 hr at room temperature. After rinsing in phosphate-buffered saline, the sections were visualized by means of a LSAB kit (Dako, Glostrup, Denmark), which employed a biotinylated secondary antibody and horseradish-streptavidin conjugate. AEC was used as a chromogenic substrate. Sections were counterstained very briefly in Mayer's hematoxylin.

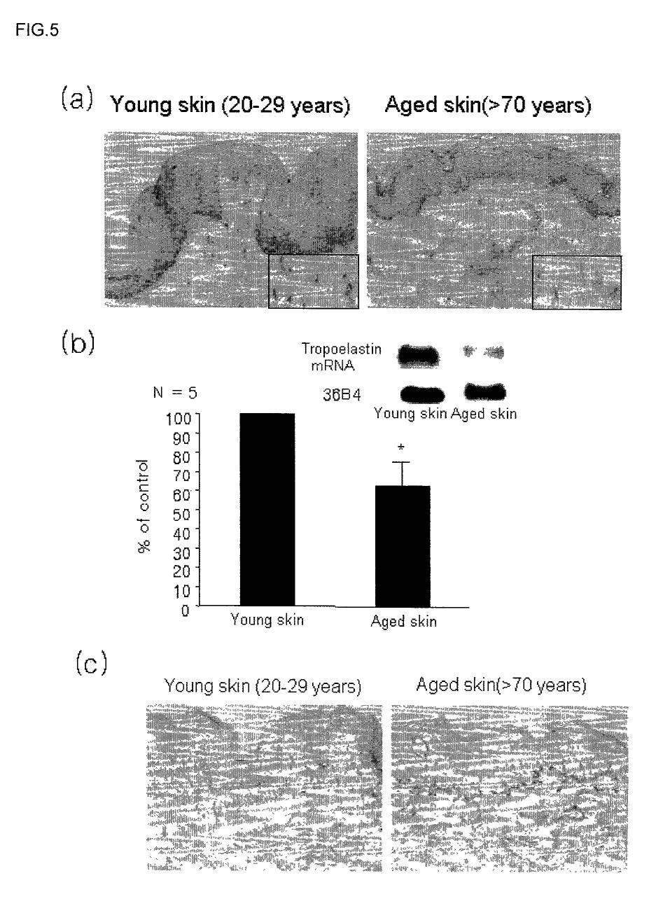

FIG. 5 shows expression of tropoelastin mRNA and protein in keratinocytes and fibroblast according to aging. Tropoelastin mRNA was measured by (a) in situ hybridization with an antisense probe, (b) northern blot analysis, and elastic fibers were stained by (c) immunohistochemical stain. In the young skin, as measured by in situ hybridization (n=5), tropoelastin mRNA was strongly expressed in the dermal fibroblasts but not in the keratinocytes. However, in aged skin, there was a dramatic decrease in tropoelastin mRNA expression in the fibroblasts and there was no expression in the keratinocytes (n=5). The “b” of FIG. 5 shows tropoelastin mRNA extracted from fibroblast in aged skin and young skin, and the graph shows relative concentration of band on gel. The tropoelastin mRNA levels, measured by northern blot analyses, were significantly lower in aged skin (n=5) by an average of 40%, when compared with young skin (n=5)

By immunohistochemical staining, the oxytalan fibers were found to be significantly lower in aged buttock skin, but the elastic fibers in the upper and mid-dermis became thicker and more fragmented, compared with those of young skin (c).

### EXAMPLE 3

Tropoelastin Expression by Chronic UV Irradiation in Photoaging

Specimens of both forearm and upper-inner arm skin from elderly persons (>70 years) were obtained. In the result of in situ hybridization, minimal amounts of tropoelastin mRNA in the fibroblasts were detected in the sun-protected skin (n=5) and there was no tropoelastin mRNA expression in the keratinocytes. However, in sun-exposed skin (n=5), tropoelastin mRNA expression increased significantly not only in the fibroblasts but also in the keratinocytes, when compared with the sun-protected skin of the same individuals.

To quantify the tropoelastin mRNA level, the total RNA was extracted directly from the punch biopsy specimens of both forearm and upper-inner arm skin from elderly subjects (>70 years). The tropoelastin mRNA levels were determined by northern blot analysis. The forearm skin demonstrated increased tropoelastin mRNA expression, when compared with the sun-protected upper-inner arm skin from the same individual (n=5).

Also, specimens of both forearm and upper-inner arm skin were stained by immunohistochemical stain. In the dermis of the sun-protected skin, a smaller amount of oxytalan fibers and more fragmented elaunin fibers were observed. However, in sun-exposed skin, there were few disrupted oxytalan fibers in the papillary dermis and large amounts of elastotic material in the reticular dermis (n=5).

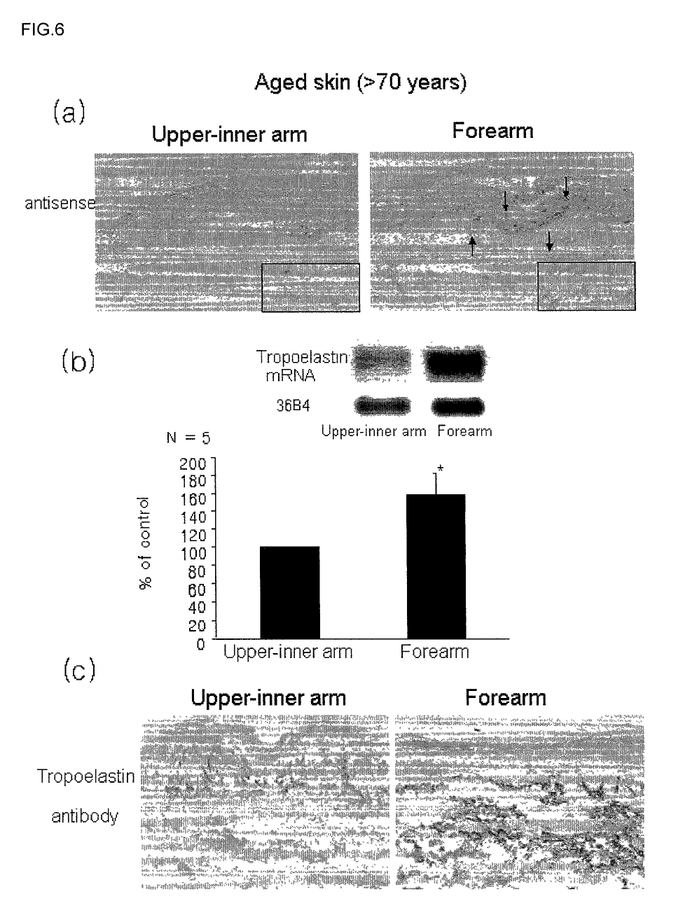

FIG. 6 is picture showing expression of tropoelastin mRNA and protein in the sun-protected skin, forearm and sun-exposed skin, upper-inner arm. Tropoelastin mRNA was detected by (a) in situ hybridization and (b) northern blot analyses. Elastic fibers were stained by immunohistochemical stain.

### EXAMPLE 4

Induction Method of Tropoelastin Expression in Elderly Persons

0.1% of the retinoic acid and control material was applied to both forearm and upper-inner arm skin from elderly persons (>70 years, n=3) for 15 days by an airtight method, and the skin of both were obtained. The control materials contain 95% ethanol/propylene glycol (7:3) and 0.05% butylated hydroxy toluene. Skin of both were stained by tropoelastin antibody

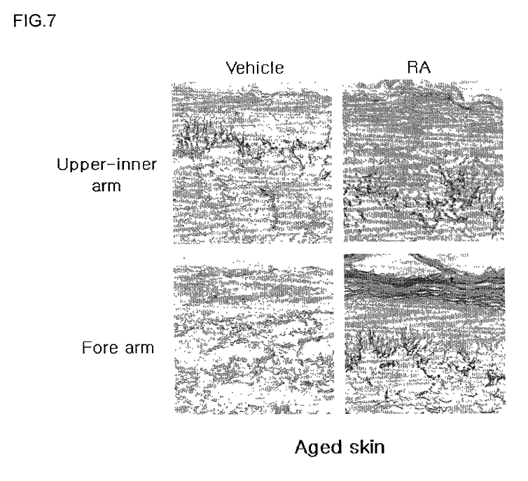

FIG. 7 is a picture showing expression of tropoelastin in retinoic acid treated aged skin. The aged skin is sun-exposed skin, forearm and sun-protected skin, upper-inner arm. In tissue that was treated with control material, the amount of oxytalan was decreased and there was no tropoelastin expression in skin of both the forearm and upper-inner arm. But in tissue that was treated with 0.1% retinoic acid, tropoelastin expression was increased in skin of both the forearm and upper-inner arm, and the length and number of oxytalan fibers was increased.

As shown above, UV induces elastin expression in human epithelial cells, keratinocytes. The composition for controlling elasticity of skin induces overexpression of tropoelastin due to UV irradiation, acts on maintenance of elasticity of skin and inhibits skin aging by preventing accumulation of elastotic material. Also, in aged skin, the composition for controlling elasticity of skin stimulates elastin expression in keratinocytes and increases elasticity of skin and inhibits skin aging while increasing the length and number of oxytalan fibers.

What is claimed is:

1. A method for controlling elasticity of skin comprising the step of using a compound that inhibits or induces expression of tropoelastin mRNA or protein in keratinocytes.

2. A method for controlling elasticity of skin according to claim 1, wherein said compound that inhibits expression of tropoelastin mRNA or protein prevents photoaging by inhibiting accumulation of elastotic materials.

3. A method for controlling elasticity of skin according to claim 1, wherein said compound that induces tropoelastin mRNA or protein, facilitates elastin protein expression in Keratinocytes.

4. A method for controlling elasticity of skin according to claim 1, wherein said compound that induces or inhibits tropoelastin mRNA or protein is selected from the group consisting of natural extracts, antioxidants, hormones, retinoid, vitamins and their mixture.

5. A method for controlling elasticity of skin according to claim 4, wherein said natural extracts are selected from the group consisting of green tea extract and soybean extract.

6. A method for controlling elasticity of skin according to claim 4, wherein said antioxidants are selected from the group consisting of vitimin C, N-acetyl cystein (NAC) and glutathione.

7. A method for controlling elasticity of skin according to claim 4, wherein said hormones are selected from the group consisting of estrogen, DHEA (dehydroepiandro sterone) and testosterone.

8. A method for controlling elasticity of skin according to claim 4, wherein said vitamins are selected from the group consisting of vitamin C, tocopherol and vitamin D.

9. A method for controlling elasticity of skin according to claim 4, wherein said retinoid is selected from the group consisting of isomers of retinoic acid, tretinoin, retinol, retinaldehydes, their salt compounds and their ester compounds.

10. A composition for controlling elasticity of skin involved in the regulation of elasticity of skin by inhibition or induction of tropoelastin mRNA or protein in human epithelial cells, keratinocytes.

11. A composition for controlling elasticity of skin according to claim 10, wherein said compound that induces or inhibits tropoelastin mRNA or protein is selected from the group consisting of natural extracts, antioxidants, hormones, retinoid, vitamins and their mixture.

12. A composition for controlling elasticity of skin according to claim 10, wherein said natural extracts are selected from the group consisting of green tea extract and soybean extract.

13. A composition for controlling elasticity of skin according to claim 10, wherein said antioxidants are selected from the group consisting of vitimin C, N-acetyl cystein (NAC) and glutathione.

14. A composition for controlling elasticity of skin according to claim 10, wherein said hormones are selected from the group consisting of estrogen, DHEA (dehydroepiandro sterone) and testosterone.

15. A composition for controlling elasticity of skin according to claim 10, wherein said vitamins are selected from the group consisting of vitamin C, tocopherol and vitamin D.

16. A composition for controlling elasticity of skin according to claim 10, wherein said retinoid is selected from the group consisting of isomers of retinoic acid, tretinoin, retinol, retinaldehydes, their salt compounds and their ester compounds.
